(19) United States
(12) Patent Application Publication
(10) Pub. No.: US 2020/0168307 A1
(43) Pub. Date: May 28, 2020

(54) METHOD AND SYSTEM FOR ACCESSING ELECTRONIC MEDICAL AND HEALTH RECORDS BY BLOCKCHAIN

(71) Applicant: TAIPEI MEDICAL UNIVERSITY, Taipei City (TW)

(72) Inventors: Ray-Jade Chen, New Taipei City (TW); Yu-Sheng Lo, New Taipei City (TW); Lan-Ying Kang, Kaohsiung City (TW); Christine Chen, San Francisco, CA (US); Yi-Chen Tseng, New Taipei City (TW)

(21) Appl. No.: 16/699,007

(22) Filed: Nov. 28, 2019

(30) Foreign Application Priority Data
Nov. 28, 2018 (TW) 107142494

(51) Int. Cl. G16H 10/60; H04L 9/32; G06F 16/2458; G06F 16/248; G06F 16/22

(52) U.S. Cl. CPC G16H 10/60; H04L 9/3242; H04L 2209/38; G06F 16/248; G06F 16/22; G06F 16/2477

(57) ABSTRACT

Provided herein is to a blockchain-implemented method and a system for accessing electronic medical and health records by blockchain. The method is operated in a server system that is used to manage one or more medical-health record databases and users' blockchain IDs. The plurality of medical-health record databases can be set by different medical-health institutions. The server system identifies the user's blockchain ID when it receives a request from a terminal device. The user uses a query interface to generate a query command. The server system then obtains one or more medical-health record indexes via blockchain verification according to the user's blockchain ID and the query command. The medical-health records corresponding to the indexes can therefore be obtained after querying the database.

### CROSS-REFERENCE TO RELATED PATENT APPLICATION

This application claims the benefit of priority to Taiwan Patent Application No. 107142494, filed on Nov. 28, 2018. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The disclosure is related to a technology for accessing electronic medical and health records, and more particularly to a method and a system for managing creation and querying of the electronic medical and health records by a blockchain technology.

### BACKGROUND OF THE DISCLOSURE

Traditional medical institutions generally store patient data by means of paper with manually created indexes. If there is a need to access the patient data, the patient data can be found according to the indexes. The patient data can be used as a reference for follow-up diagnosis and treatment.

Such conventional paper records can also be digitalized to electronic medical records. The electronic medical records are beneficial to circulation and portability. The electronic medical records allow a doctor to deal with a patient's condition more comprehensively and correctly since it is convenient for the doctor to refer to the patient's records in other medical departments.

It is common practice for the traditional medical institutions to store their own medical records with no way to share them. Through national health insurance, the medical records are often stored in specific server systems of government agencies for centralized management. However, the traditional framework of the server systems may harbor security concerns such as data leakage or malicious tampering, and may not allow the public or the patients to perform secure queries due to any flaws in the security mechanism.

### SUMMARY OF THE DISCLOSURE

The disclosure relates to a blockchain-based method for accessing electronic medical and health records, and a system thereof. Blockchain has features such as being secure, correct and decentralized, allowing the system to create indexes for the electronic medical and health records. Each of the indexes links to its corresponding electronic medical and health records. The records may include electronic medical records (EMR) and electronic health records (EHR). The electronic medical and health records can thus be queried and established with proper consideration to security and correctness.

According to one of the embodiments of the disclosure, the method for accessing the electronic medical and health records is operated in a server system. The server system has a database. The method allows a user or personnel of a medical-health institution to obtain blockchain ID and key by a registration process. When the server system receives a connection request sent from a terminal device, the server system verifies the blockchain ID of the user of the terminal device. After that, the server system receives a query command from the terminal device, and acquires one or more indexes for medical and health records by performing blockchain verification according to the user's blockchain ID and a query command. When querying the database, one or more medical and health records can be obtained.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic diagram depicting a system framework for accessing electronic medical and health records using blockchain technology according to one embodiment of the disclosure.

FIG. 2 is a schematic diagram depicting a system framework for accessing electronic medical and health records using blockchain technology in another embodiment of the disclosure;

FIG. 3 shows a flow chart describing a process in a terminal device in a method for accessing electronic medical and health records in one embodiment of the disclosure;

FIG. 4 shows a flow chart describing a process in a server system in a method for accessing electronic medical and health records in one embodiment of the disclosure;

FIG. 5 shows a flow chart describing a process for medical staff in a method for accessing electronic medical and health records in one embodiment of the disclosure;

FIG. 6 is a schematic diagram describing a process in a user device in the method for accessing electronic medical and health records in one embodiment of the disclosure; and

FIG. 7 is a schematic diagram describing another process in the user device in the method according to one embodiment of the disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The disclosure relates to a method for accessing electronic medical and health records (hereinafter ‘EMR/HER’) and a system thereof. The method in particular utilizes blockchain technology to create indexes for indicating the EMR/EHR records for linking with a medical-health record database. The indexes are converted into decentralized records by the blockchain technology. The decentralized records allow an authorized user to access the electronic EMR/EHR records based on his level of authorization. The system utilizes the features of blockchain such as security, correctness and decentralization to create the indexes for the electronic EMR/EHR records. The EMR/EHR records can also be queried and established in the database with consideration to security and correctness. The above-mentioned electronic EMR/EHR records can be categorized into electronic medical records (EMR) that are generated in hospitals, medical institutions or health examination centers, and electronic health records (EHR) that are generated by inspection devices. The records in the database can also include both EMRs and EHRs, and can also be the medical records or health records of patients.

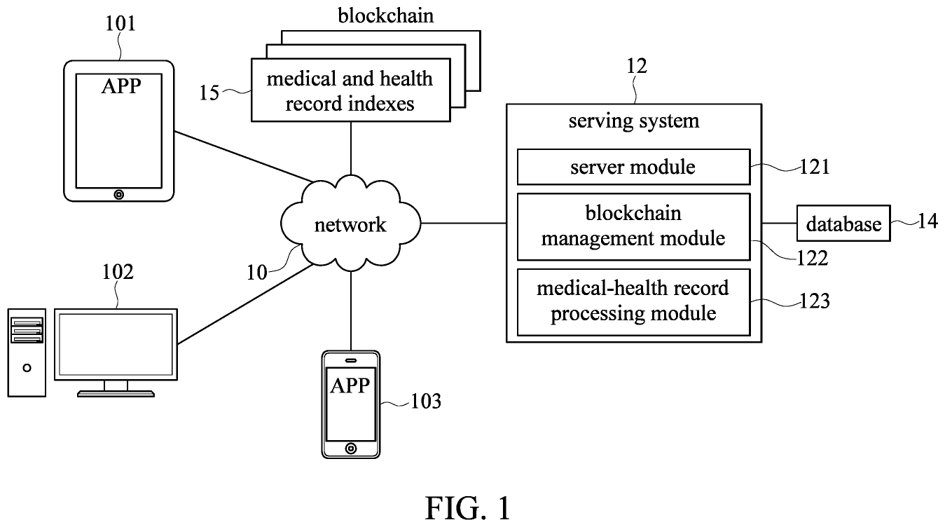

Reference is made to FIG. 1, showing a system framework that implements a system for accessing electronic EMR/EHR records according to one embodiment of the disclosure.

The main components of the system for accessing the electronic EMR/EHR records include a computer-implemented server system 12. The server system forms a cloud platform via a network 10. The cloud platform combines one or more medical-health institution systems. The server system 12 may have its own database 14 or combines one or more medical-health record databases of the one or more medical-health institution systems. The medical-health institution can be a medical institution or a health examination center. In addition to the equipment at the server end, the system also provides software programs stored in various terminal devices 101, 102 and 103. The software program can be operated in a mobile device or a computer terminal. The software program can be successfully executed after an authentication process is passed, e.g. fingerprint verification, password validation or face recognition. When the software program is executed by a processor of one of the terminal devices (101, 102, 103), the terminal device connects with the server system 12 via the network 10. A query command can be generated through a user interface. The query command can be adapted to retrieve the indexes for the electronic EMR/EHR records in a blockchain network 15.

The server system 12 provides functionalities that are implemented by hardware and software. The functionalities can be embodied by a server module 121, a blockchain management module 122 and a medical-health record processing module 123 in the server system 12. The server module 121 provides services such as establishing connectivity for the terminal devices 101, 102 and 103 and initiating a user interface allowing the terminal devices to connect with the server system 12 via Internet protocol, e.g. HTTP.

The blockchain management module 122 is responsible for managing blockchain IDs of users. The blockchain management module 122 allows the users to register blockchain IDs and the related certificates, and to acquire public keys, private keys and a password pertinent to the keys. The blockchain management module 122 issues the indexes for electronic EMR/EHR records and verifies the services for acquiring the indexes. For example, the blockchain management module 122 verifies the blockchain ID of a user who initiates the connection. The system therefore provides services according to the user's identity. Further, the blockchain management module 122 stores the indexes for indicating the electronic EMR/EHR records by decentralized ledgers or addresses of a specific blockchain network through an encryption algorithm. Further, a medical-health record processing module 123 is responsible for processing the procedure to establish the database 14 for the electronic EMR/EHR records in the server system 12. The module 123 then creates the indexes for the records. In one embodiment of the disclosure, the blockchain management module 122 manages the pubic keys for the users or patients. When a new record is created by a patient, the medical-health record processing module 123 acquires the public key after receiving the record. The key allows the system to encrypt the new electronic EMR/EHR record, and to create an index for the electronic EMR/EHR record. The encrypted record is then stored into the database 14 through the module 123. The index is uploaded to the blockchain network 15 by a decentralized ledger protocol using an encryption algorithm, and the index forms one of the records in the blockchain network 15.

Through the indexes over the blockchain network 15, the server system 12 is able to access the medical-health record database of other medical-health institution systems. In addition, the server system 12 allows its users to create electronic EMR/EHR records and forms the indexes that link to the records by the blockchain technology. The main components of the electronic EMR/EHR records stored in the database 14 are patient IDs, timestamps with respect to the records, details related to the records, and queryable database addresses that are linked with the indexes.

The terminal devices 101, 102 and 103 are such as the regular user-end computer devices. A program process performed in the terminal device initiates a user interface for assisting the user to join the blockchain network 15 provided by the system. After that, the user can retrieve the records via the user interface and access the records in accordance with the level of authorization according to the user's blockchain ID. The medical staff or care personnel may have the authorization to access the records of patients over the blockchain network in accordance with his blockchain IDs, and then to create the EMR/EHR record after diagnosis and treatment.

In the method for accessing the electronic EMR/EHR records, the following process allows a regular user to look up the records, and the medical staff to create the records.

It should be noted that the blockchain technology is incorporated into the method for accessing the EMR/EHR records not only for assisting the users to acquire their blockchain IDs and corresponding keys, but also for providing service to look up the records. The indexes with respect to the records are created and stored over the decentralized nodes of blockchain network 15. Further, the indexes can also be stored to designated host nodes. Each index over the blockchain network 15 records a corresponding database address. For example, the database address indicates an address for each record in the database, or to an address for a file in an archive. The index acts as a virtual link that is provided as a link to the actual record/file stored in the database at a medical-health institution system. Therefore, the blockchain-based index has advantages of being secure, tamper-proof, and convenient when making queries since the user would need to be firstly verified by the server system 12 using his blockchain ID, obtain the authorization to query the indexes of records, and then retrieve the actual data in the database 14.

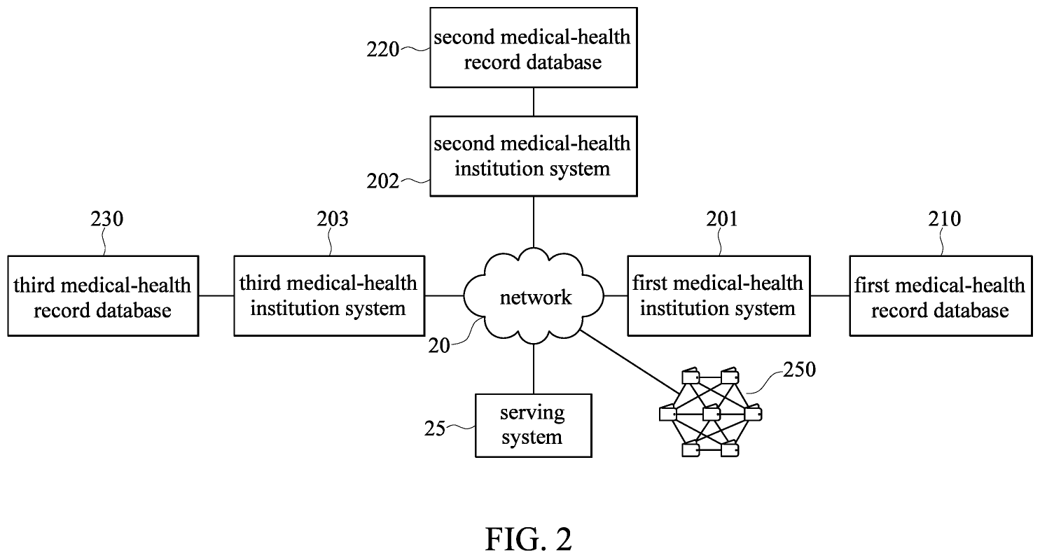

Reference is made to FIG. 2 that shows another system framework for managing the electronic EMR/EHR records using a blockchain technology in one embodiment of the disclosure.

A server system 25 forms a cloud system that connects with one or more medical-health institution systems (201, 202, and 203). The server system 25 links to each of the medical-health record databases (210, 220, 230) of each of the medical-health institution systems (201, 202, 203), or is constituted by the medical-health record databases (210, 220, 230) of the medical-health institution systems (201, 202, 203).

In the present embodiment, the server system 25 links with a plurality of medical-health institution systems (201, 202, and 203). The server system 25 implements a cloud platform and establishes a cloud database that is composed of the medical-health record databases (210, 220, 230) provided by the medical-health institutions. The plurality of medical-health institution systems (201, 202, and 203) form the nodes in a blockchain network. In an exemplary example, the server system 25 connects to a first medical-health institution system 201, a second medical-health institution system 202 and a third medical-health institution system 203 via a network. The system initiates a blockchain 250 for sharing the indexes for the electronic EMR/EHR records. These medical-health institution systems (201, 202, and 203) form the nodes of blockchain 250. The systems (201, 202, and 203) also have their proprietary medical-health record databases that may be represented by the first medical-health record database 210, the second medical-health record database 220 and the third medical-health record database 230.

The system adopts a blockchain technology to record indexes for electronic EMR/EHR records. The medical-health institutions share the indexes for EMR/EHR records in the blockchain 250. Each of the indexes for EMR/EHR records links with a corresponding EMR/EHR record stored in respective ones of the medical-health record databases (210, 220, and 230). An end user such as a patient or a medical staff is able to access the records by the blockchain 250 via a network 20. The system for acquiring the electronic EMR/EHR record integrates various EMR/EHR records from different locations. The system allows every patient to access his EMR/EHR records created at different medical-health institutions. Further, the medical staff can obtain complete EMR/EHR records rather than only part of the records or the records from a single source as may be the case with the conventional technology.

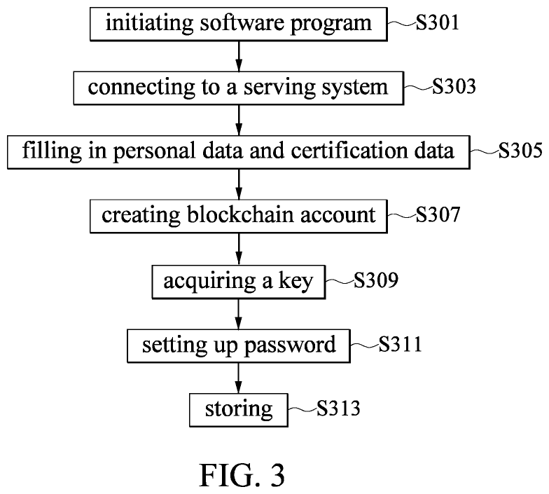

FIG. 3 to FIG. 5 respectively describe a process operated in a user device for accessing electronic EMR/EHR records, a process in the system for managing queryable EMR/EHR records, and a process in a device of medical staff for managing EMR/EHR records in the embodiments of the disclosure.

FIG. 3 shows a flow chart illustrating the process for a user to register to a system service. In step S301, the user manipulates a terminal device to perform a software program provided by a system, or a specific program. The terminal device is then connected with a server system (step S303). The server system submits a request for activating a user device by the software program. In step S305, a user can fill in his personal data and verification data using the software program.

In step S307, the system assists the user to create his blockchain account and ID according to his personal data. In step S309, the system also generates a pair of keys including a private key and a public key that allow the user to decrypt and encrypt the blockchain-based data. In step S311, the system may request the user to set up a password for the private key via a user interface. It should be noted that the aspect of the password can be replaced by other aspects of authentication, for example, a fingerprint verification or facial recognition. After the user registration process to the system service, the requisite data such as the key is stored into the terminal device (step S313).

In the process of creating the blockchain ID, the system requires the user to install a corresponding software program and provide the verification data. In the meantime, the user can choose a device to participate in the verification process. Further, the software program is configured to be bound with the device for strengthening the security. The device binding can prevent the keys or the identity data from being stolen. According to the settings made during this registration process, a regular user, e.g. the patient, only has permission to access his own EMR/EHR records.

Still further, the medical staff also needs to obtain his own blockchain ID for accessing the EMR/EHR records in the blockchain. A software program is installed in the device used by the medical staff. When the medical staff is authorized by the patient, the medical staff is able to access the patient's EMR/EHR records. In an exemplary example, the medical staff can obtain the patient's authorization by scanning an authorization code, e.g. QR Code, provided by the patient. After that, the medical staff can access the records after a verification process according to the patient's authorization and his blockchain ID. Specifically, the medical staff obtains indexes for electronic EMR/EHR records and then accesses the content in the records.

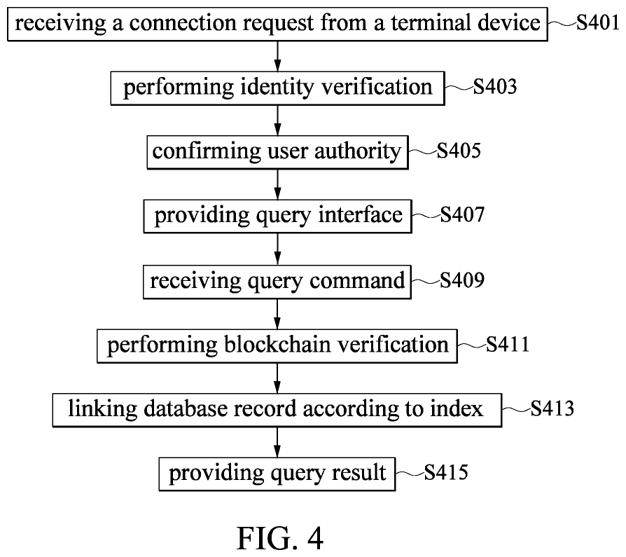

FIG. 4 shows a flow chart describing the method for accessing the EMR/EHR records in one embodiment of the disclosure.

In an initial step S401, a server system receives a connection request generated by a terminal device via a user interface. The connection request includes a user's verification data that may correspond to the user's blockchain identification data. Therefore, the server system performs an identity verification process (step S403), and verifies the user authorization according to the user's blockchain ID (step S405).

The system then initiates a query interface for the user to query the records through a software procedure (step S407). The user inputs a query criterion such as a date range for obtaining the records within the date range. The records are such as diagnosis data, EMR/EHR data, and/or examination reports. If the user adds query criterions such as a specific medical institution, a doctor name or a specific department, a query command is generated and transmitted to the server system (step S409).

The server system requires the blockchain nodes to verify the records corresponding to the query command based on the user's blockchain ID and the query command (step S411). The server system can therefore obtain one or more indexes for the electronic EMR/EHR records. The indexes allow the server system to query the records in a database and obtain the actual data of the records (step S413). The result is then transmitted to the terminal device via a network (step S415). The indexes are tamper-proof since the result is verified by a plurality of blockchain nodes in the process for acquiring the indexes. In the terminal device, a key is required to decrypt the query result so as to obtain the actual data of the records.

In one of the embodiments, when the server system obtains the indexes for the records, the system can link to the actual data of records in the database according to the indexes. The actual data are such as the electronic medical records, health records and/or related files. The one or more indexes form a query result in the system. The query result is provided to the terminal device. Using the one or more indexes, the terminal device can obtain the actual data of the records.

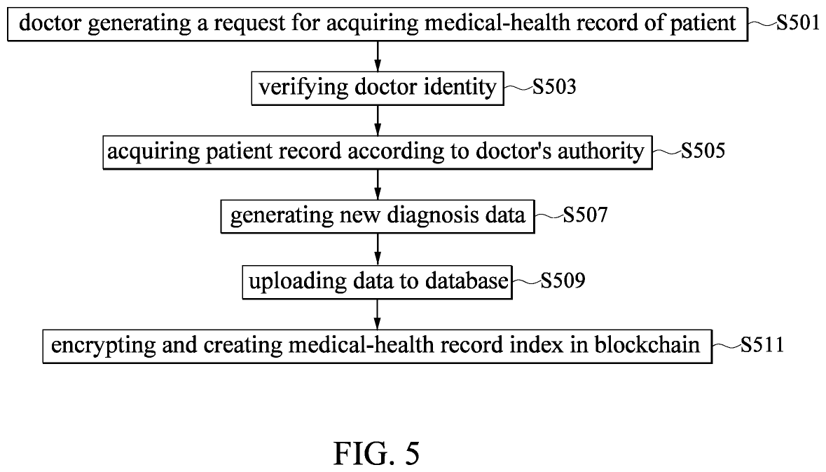

FIG. 5 shows a flow chart that describes a process operated in a device of the medical staff for establishing the EMR/EHR records in one embodiment of the disclosure.

In step S501 of the process, a doctor-end device generates a request for acquiring a medical-health record of a patient. For example, when a medical staff, e.g., the doctor, inquires the patient about his condition, the medical staff may need to obtain the patient's EMR/EHR records. After obtaining the patient's authorization, the medical staff operates the device to generate a request for accessing the EMR/EHR records. When the server system receives the request, in step S503, the server system firstly verifies the medical staff's identity. The server system can verify the blockchain ID of the medical staff and the records in the request according to the verification data sent by the medical staff. The verification data can include a blockchain ID, the patient's identity, and authorization data. Next, the server system acquires the EMR/EHR records in accordance with the authorization related to the request (step S505).

It is possible to generate new diagnosis data, medical data, and/or examination reports when the medical staff conducts the inquiry and diagnosis (step S507). The newly-added electronic EMR/EHR records can also be encrypted using the patient's public key and uploaded to the database (step S509). In step S511, the indexes for indicating the medical-health records in the database are created. The indexes are then encrypted to form the records distributed over the blockchain network.

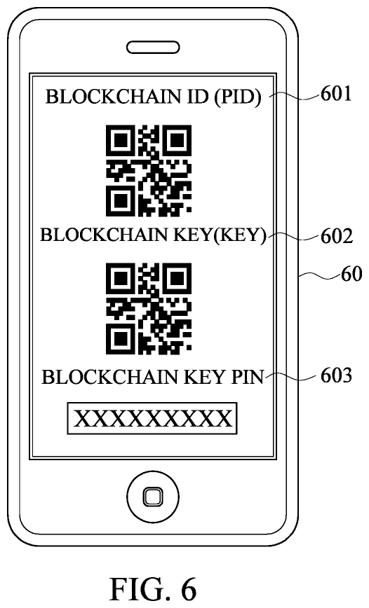

FIG. 6 shows a schematic diagram depicting a screenshot of a user terminal device in one embodiment of the disclosure.

The figure schematically shows a terminal device 60 performing blockchain verification, in which a blockchain ID (PID) 601 is shown. The blockchain ID can be represented by a QR Code used for verification. Through the QR Code or the like representing the blockchain ID, other users can obtain authorization from the patient to access the patient's EMR/EHR records. The authorized user can firstly obtain the indexes for the EMR/EHR records from the blockchain by decrypting the blockchain records using a blockchain key (KEY) 602. The software process running in the device will require the user to key in a blockchain key PIN 603 to retrieve the private key so as to decrypt the blockchain records.

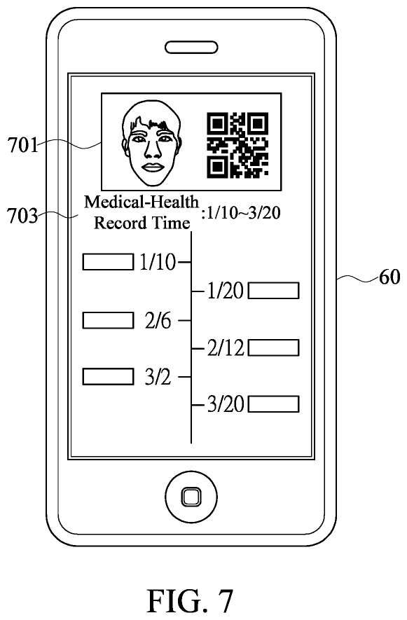

Reference is made to FIG. 7, which shows a schematic diagram depicting a screenshot showing the electronic EMR/EHR records obtained from the database.

In the diagram, a personal blockchain data 701 and the EMR/EHR records (703) within a time period obtained by the terminal device 60 after acquiring the indexes are shown. The shown records also include information such as a patient ID, timestamps, and other contents in the records. The terminal device 60 displays the one or more records within a time period via a user interface. In an exemplary example, the records shown on an APP screen of the device include the recent record indexes or record indexes within a time period designated by the user that are arranged on the screen in a chronological order.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for accessing electronic medical and health records, operated in a server system with a database, comprising:
receiving a connection request from a terminal device;
verifying a blockchain ID of a user of the terminal device;
receiving a query command from the terminal device; and
obtaining one or more indexes for medical and health records after performing blockchain verification according to the blockchain ID of the user and the query command, wherein the one or more indexes form a query result, and the one or more indexes correspond to one or more electronic medical and health records of the database.

2. The method according to claim 1, wherein the one or more electronic medical and health records are created within a period of time, and the one or more electronic medical and health records are displayed on the terminal device in a time sequence when transmitting the records to the terminal device.

3. The method according to claim 1, wherein the electronic medical and health records are created by steps of:
the server system receiving a request for accessing the electronic medical and health records from a terminal device of a medical staff;
the server system verifying a blockchain ID of the medical staff;
the server system receiving a new diagnosis data, medical data, and/or examination report from the terminal device of the medical staff;
creating a new electronic medical and health record in the database; and
forming a corresponding new index for the new electronic medical and health record, which forms an index in the blockchain after encryption of the new electronic medical and health record.

4. The method according to claim 1, wherein the server system initiates a user interface through a software program executed in the terminal device, and receives blockchain ID data of the user via the user interface so as to verify the blockchain ID; and a query interface is provided for receiving the query commands.

5. The method according to claim 4, wherein the one or more electronic medical and health records are created within a period of time, and the one or more electronic medical and health records are displayed on the terminal device in a time sequence when transmitting the records to the terminal device.

6. The method according to claim 4, wherein the electronic medical and health records are created by steps of:
the server system receiving a request for accessing the electronic medical and health records from a terminal device of a medical staff;
the server system verifying a blockchain ID of the medical staff;
the server system receiving a new diagnosis data, medical data, and/or examination report from the terminal device of the medical staff;
creating a new electronic medical and health record in the database; and
forming a corresponding new index for the new electronic medical and health record, which forms an index in the blockchain after encryption of the new electronic medical and health record.

7. The method according to claim 1, wherein the server system forms a cloud system that connects with one or more medical-health institution systems, and the database of the server system links with every medical-health record database corresponding to each of the medical-health institution systems, or is composed of a plurality of medical-health databases of each of the medical-health institution systems.

8. The method according to claim 7, wherein the index for each of the electronic medical and health records in the blockchain records a database address that is used to link to an electronic medical and health record in the electronic medical and health record database of the medical-health institution system.

9. The method according to claim 8, wherein the one or more electronic medical and health records are created within a period of time, and the one or more electronic medical and health records are displayed on the terminal device in a time sequence when transmitting the records to the terminal device.

10. The method according to claim 9, wherein the records are created by steps of:
the server system receiving a request for accessing the electronic medical and health records from a terminal device of a medical staff;
the server system verifying a blockchain ID of the medical staff;
the server system receiving a new diagnosis data, medical data, and/or examination report from the terminal device of the medical staff;
creating a new electronic medical and health record in the database; and
forming a corresponding new index for the new electronic medical and health record, which forms an index in the blockchain after encryption of the new electronic medical and health record.

11. A system for accessing electronic medical and health records, comprising:
a server system having a database; and
a software program stored in a terminal device, when the software program is executed by a processor of the terminal device, the terminal device connects with the server system, generates a query command, and retrieves one of the indexes for the electronic medical and health records in blockchain;
wherein the server system operates a method using blockchain to manage queryable electronic medical and health records, comprising:
receiving a connection request transmitted by the terminal device;
verifying a blockchain ID of a user of the terminal device;
receiving the query command from the terminal device;
obtaining one or more indexes for one or more electronic medical and health records after performing blockchain verification according to the blockchain ID and the query command; and
forming a query result using the one or more indexes for one or more electronic medical and health records, wherein the one or more indexes correspond to one or more electronic medical and health records in the database.

12. The system according to claim 11, wherein the one or more electronic medical and health records are created within a period of time, and the one or more electronic medical and health records are displayed on the terminal device in a time sequence when transmitting the records to the terminal device.

13. The system according to claim 11, wherein the server system forms a cloud system that connects with one or more medical-health institution systems, and the database of the server system links with every medical-health record database corresponding to each of the medical-health institution systems, or is composed of a plurality of medical-health databases of each of the medical-health institution systems.

14. The system according to claim 13, wherein the index for the medical and health record records a database address for linking with electronic medical and health records or files in the medical-health record database of the medical-health institution system.

15. The system according to claim 14, wherein the one or more electronic medical and health records are created within a period of time, and the one or more electronic medical and health records are displayed on the terminal device in a time sequence when transmitting the records to the terminal device.
